(19) United States
(12) Patent Application Publication
(10) Pub. No.: US 2017/0266105 A1
(43) Pub. Date: Sep. 21, 2017

(54) TOPICAL DEODORANT MADE WITH 100% VEGAN INGREDIENTS

(71) Applicant: Michael Strickland, New Port Richey, FL (US)

(72) Inventor: Michael Strickland, New Port Richey, FL (US)

(21) Appl. No.: 15/461,880

(22) Filed: Mar. 17, 2017

Related U.S. Application Data
(60) Provisional application No. 62/310,682, filed on Mar. 19, 2016.

(51) Int. Cl. A61K 8/97; A61K 8/02; A61K 8/92; A61Q 15/00; A61K 8/73

(52) U.S. Cl. CPC A61K 8/97; A61Q 15/00; A61K 8/732; A61K 8/922; A61K 8/0216

(57) ABSTRACT

The present invention discloses a novel and improved composition and method of preparation of organic topical deodorant. The invention utilizes all readily available natural vegan ingredients for formulation of the deodorant product that results in effective elimination of body odor and retains good structural integrity without drying out or becoming rancid. Present invention achieves the main objectives by creating a unique composition comprised of pure corn starch, unbleached organic all-purpose flour, candelilla wax, virgin coconut oil, raw unfiltered organic apple cider vinegar and an optional fragrance. The disclosed ingredients are cooked in a double boiler at a certain temperature and in a manner that yields the claimed deodorant compound. With the process of this invention coconut oil can be substituted with other natural oils and fragrance can be altogether eliminated from the process without compromising deodorant's efficacy. Not only is the final deodorant compound safe for topical use, but also due to its unique combination of natural ingredients, no harmful by-products are formed during the process of preparation. This method of preparation thus gives the final product an environmental advantage over the other similar products known in the prior art.

### TECHNICAL FIELD OF INVENTION

The invention broadly relates to a deodorant composition and method of its preparation, using purely natural vegan ingredients. In particular, the invention relates to an organic deodorant that combines the fewest number of ingredients which safely and effectively eliminates underarm odor.

### BACKGROUND OF THE INVENTION

Hircus, or underarm odor, violates the tacit rules of etiquette in America and many other parts of the world. Hence, the daily use of deodorant is essential in the maintenance of personal hygiene. Unfortunately, many deodorants and antiperspirants use chemical compounds such as aluminium, triclosan, propylene glycol, parabens, and esters in order to create synthetic fragrances for preventing sweating and body odor. Sadly, studies have demonstrated that these unnatural deodorants produce harmful side effects. In fact, a variety of health disorders including breast cancer, Alzheimer's, bone disorders, kidney problems, skin irritations, and allergies have been linked to the use of deodorants that contain these harmful chemicals. Consequently, there has been a dire need for a product that is natural and that provides a healthy alternative to non-natural deodorants. The chemicals used in non-natural deodorants readily enter the body by absorption through the skin, and those chemicals that the human body cannot assimilate or eliminate become toxic. Many of today's health-conscious consumers are switching to natural deodorants because natural deodorants effectively eliminate malodor without exposing them to the health risks associated with the use non-natural deodorants.

Natural deodorants have addressed the identified concerns, but the common complaints regarding natural deodorants are their fragility, their instability, and that they simply do not work. Another common problem experienced by those who use a natural deodorant that contains sodium bicarbonate (baking soda) is that its use tends to cause skin irritations.

The present invention seeks to overcome these common issues. The deodorant composition in accordance to the present invention, made from specific ingredients through a unique process for combining them, has realized the invention's primary objective of creating a simple and safe all-natural deodorant that effectively eliminates underarm odor.

Major pitfalls reported for natural deodorants include inefficacy in eliminating odor, short shelf life, and limited ability to maintain structural stability. Attempts to overcome these issues invariably involve adding more chemical compositions to the natural ingredients, thus making the composition partially non-natural.

Although well-known for its inherent characteristic of naturally eliminating body odor, sodium bicarbonate often causes skin irritation, as stated, and generally compromises the structural integrity of natural deodorants. Moreover, since chemicals are more readily absorbed into the body through the armpits than almost any other part of the body, deodorants that use sodium bicarbonate could pose a serious health threat since high sodium intake generally causes elevated blood pressure. The present invention thus provides odor-eliminating properties without using sodium bicarbonate.

A major benefit of the invention is its variety of versions. No available prior art in the all-natural deodorant field provides multiple variations to the user. For instance, those with an aversion to coconut oil or a specific fragrance will have the option of choosing another safe and effective version of the deodorant that incorporates an alternative fragrance or an alternative natural oil.

There has been a long standing need for an organic deodorant that integrates a minimum number of natural ingredients, yet is durable, safe, and effective at eliminating underarm odor. Organic deodorants existing in prior arts fail to incorporate all these features in a single product. The present invention addresses all of the aforementioned problems identified in this field with unrivaled simplicity and effectiveness to provide a single product that overcomes these problems.

### SUMMARY OF THE INVENTION

The present invention resolves the issues and limitations identified in the prior art by creating a novel composition using a simplified method of preparation of deodorant product that combines a minimum number of readily available, all-natural ingredients. The resultant organic deodorant effectively eliminates underarm odor without causing any harmful effects to either the user's skin or health.

In one of its material embodiments, the present invention achieves the main objective by creating a unique composition comprised of pure corn starch, unbleached organic all-purpose flour, candelilla wax, virgin coconut oil, raw unfiltered organic apple cider vinegar, and optional fragrance, wherein corn starch is used for neutralizing odor and absorbing moisture; unbleached organic all-purpose flour facilitates the homogeneous blending of the corn starch within the deodorant compound; candelilla wax adds firmness to the deodorant compound even at warm atmospheric temperature due to its relatively high melting point; and coconut oil not only acts as a solvent for the candelilla wax, but also is an antibacterial, antifungal, and antiviral agent that kills odor-producing bacteria while effectively moisturizes the skin, and raw unfiltered organic apple cider vinegar thickens the deodorant compound and further enhances its antimicrobial properties. Note here that the coconut oil can be substituted with a different natural oil having similar chemical properties.

Although shea butter is a skin nourishment product whose use is well known in prior arts, it has been found that the mixing of shea butter with the other ingredients limits shelf life and results in overall loss of structural stability of the deodorant over time. In contrast, coconut oil is well known for its antibacterial properties, is an extraordinary skin soothing agent, is absorbed into the skin more readily, and has its own distinct natural fragrance. In addition to its effective moisturizing characteristic, coconut oil helps in binding all ingredients together in the present invention.

The deodorant formulation in the present invention requires precise proportions of pure corn starch and unbleached organic all-purpose flour that are mixed together to form a dry mixture; the coconut oil is then heated to 100° C., to which a specific proportion of candelilla wax and fragrance is added. Subsequent to complete dissolution of the candelilla wax within the coconut oil, the dry mixture of corn starch and unbleached organic all-purpose flour is added to the heated coconut oil mixture. Using a double-boiler, the heat of the ingredients is maintained at 100° C. while the coconut oil, candelilla wax, fragrance, and dry mixture of corn starch and unbleached organic all-purpose flour are thoroughly mixed.

During the mixing of these ingredients, the deodorant compound assumes the consistency and appearance of gravy. At this phase, the raw unfiltered organic apple cider vinegar is added to the compound. The precise amount of organic apple cider vinegar (immediately after pouring) is thoroughly mixed with the deodorant compound. The mixture immediately thickens, assuming the consistency and appearance of apple sauce. The deodorant compound is immediately removed from heat after it reaches the appearance of apple sauce. At this point the resulting deodorant compound is poured into empty deodorant bottles and is allowed to cool at room temperature for approximately 2 hours. The solid deodorant compound results after cooling and is ready for use.

All ingredients used in the present invention are readily available and can be stored at room temperature indefinitely without concern for any of the ingredients becoming rancid or losing their efficacy. Not only is the final deodorant compound safe for topical use, but also due to its unique combination of natural ingredients, no harmful by-products are formed during the process of preparation. The unique method of preparation thus gives the final product an environmental advantage over the other similar products known in the prior art.

The present invention has accomplished several objectives. First, the primary objective of the invention, to provide a topical natural deodorant composed of a minimum number of 100% natural ingredients which effectively eliminate underarm odor without exposing the user to any harmful chemicals was achieved.

Another accomplished objective was providing a natural deodorant which retains its shelf life and structural integrity for an extended period. A further objective attained was outlining a simple, reproducible, and cost-effective process for preparing the all-natural deodorant.

Objectives, features, aspects, and advantages of the present invention will be demonstrated in the following detailed description of the preferred embodiments of the invention, along with the accompanying drawings.

### BRIEF DESCRIPTION OF THE DRAWINGS

FIG. 1 is a table outlining the ingredients and its proportion used in manufacturing the deodorant product in accordance with the present invention.

FIG. 2 shows a representation of the double boiler that is required for cooking or manufacturing the desired deodorant product.

FIG. 3 is an overview of the first part of the process of the deodorant formulation

FIG. 4 is an overview of the second part of the process of manufacturing the deodorant compound that is in continuation from FIG. 3.

### DETAILED DESCRIPTION

Having described the main features of the above invention, a brief and non-limiting description of a preferred embodiment will be given in the following paragraphs with reference to the accompanying drawings.

In all the figures, like reference numerals represent like features. Further, the shape, size and number of the devices shown are by way of example only and it is within the scope of the present invention to change their shape, size and number without departing from the basic principle of the invention.

Throughout the specification, the technical terms and abbreviations are to be interpreted in the broadest sense of the respective terms, and include all similar items in the field known by other terms, as may be clear to persons skilled in the art. Restriction or limitation, if any are referenced in the specification, are solely by way of example for understanding the present invention.

All percentages, ratios, and parts used in the Specification, Examples, and Claims are by means of volume unless otherwise stated.

The description may be comprised of illustrative, preferred, and alternative embodiments of the present invention. Having thus described exemplary embodiments of the present invention, it should be noted by those skilled in the art that the disclosures are exemplary only, and that various other alternatives, adaptation, and modifications may be made within the scope of the present invention. Merely listing the steps of the method in a certain order does not necessarily constitute any limitation on the order of the steps of the method. Many modifications and other embodiments of the invention will come to mind to one skilled in the art to which this invention pertains having the benefit of the teachings presented in the description. Therefore, it is to be understood that the invention is not to be limited to the specific embodiments disclosed and that modifications and other embodiments are intended to be included within the scope of the claims. Although specific terms are employed herein, they are used in a generic and descriptive sense only and not for purposes of limitation. Accordingly, the present invention is not limited to the specific embodiments illustrated herein, but is limited only by the claims as written.

The present invention seeks to address the drawbacks associated with the existing organic topical deodorants. To achieve this purpose, the present invention discloses a novel combination of ingredients through an uncomplicated method for preparation of a natural topical deodorant that uses a minimum number of readily available ingredients to make a final product which gently eliminates underarm odor while sustaining remarkable shelf-life.

The ingredients/compositions utilized for this subject invention provide all desirable properties of a topical deodorant which, without limitation, includes effective neutralization of underarm odor without causing detriment to user's skin or health, a long-shelf life and stability, soothing fragrance, and a safe preparation method that leaves no harmful by-products detrimental to the environment.

The present invention in one of the preferred embodiments attains the objective by incorporating a unique composition comprised of pure corn starch, unbleached organic all-purpose flour, candelilla wax, virgin coconut oil, raw unfiltered organic apple cider vinegar, and an optional fragrance.

Each of the identified components has distinguishing characteristics relevant to the present invention's preparation. For instance, corn starch is noted for its odor neutralizing properties and has an excellent ability to absorb moisture. Unbleached organic all-purpose flour is embodied in order to ensure the even blending of the corn starch within the deodorant compound. Candelilla wax, which has a relatively high melting point, is the dominant ingredient used for maintaining the firmness and structural integrity of the deodorant compound, even at warm atmospheric temperature. Virgin coconut oil acts as a solvent for the candelilla wax and helps to bind all other ingredients together in formulation of the compound. Although well-known for providing effective skin nourishment, coconut oil is also desired for its antibacterial, antifungal, and antiviral properties that kill yeast and bacteria. While the addition of raw unfiltered organic apple cider vinegar thickens the deodorant compound, it also enhances its antimicrobial properties. And finally an optional natural fragrance is added to the compound.

In an alternative embodiment of this invention, coconut oil is substituted with a different natural oil that reacts well with the other ingredients. Likewise, the natural fragrance may be varied or entirely excluded.

Pure corn starch and unbleached organic all-purpose flour are combined in a mixing bowl. And the coconut oil, candelilla, and optional fragrance are heated in a double boiler in a specific manner to yield the desired deodorant compound. The resulting compound assumes and retains its solid state upon cooling.

In a detailed aspect of the present invention, the following represents the proportions by volume of the ingredients specified in relative amounts:
8 parts of pure corn starch
8 parts of unbleached organic all-purpose flour
3 parts of organic raw unfiltered apple cider vinegar
12 parts of pure coconut oil
6 parts of candelilla wax
2 part of fragrance

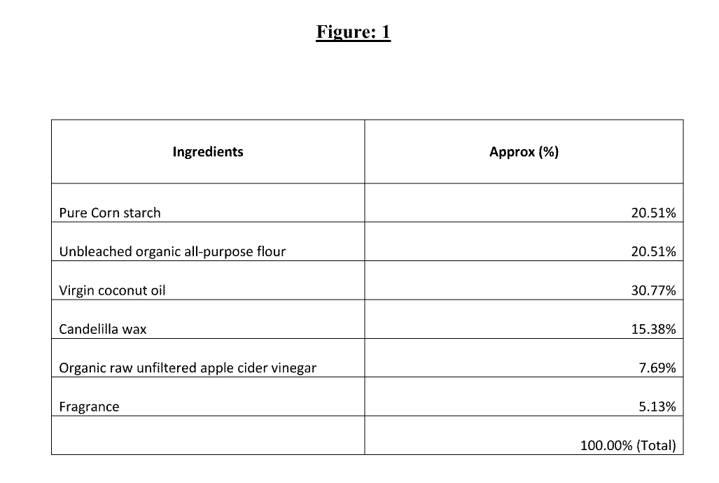

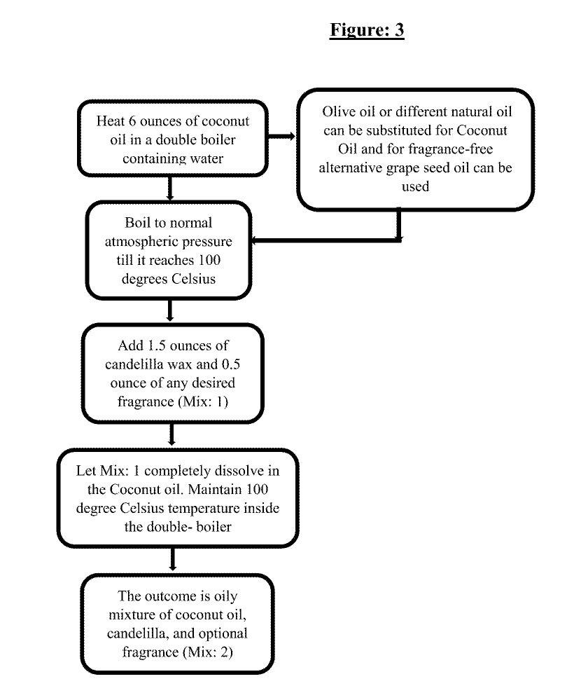

FIG. 1 shows the table outlining the percentages of the ingredients comprising the deodorant product in accordance with the present invention. A description of the manufacturing process for the desired deodorant product is described below under FIGS. 3 & 4.

The specified proportions are exemplary in conformity with the absolute amounts of the ingredients disclosed in the below mentioned steps of making the deodorant compound. Such approximation of measurement may vary depending on the application or in order to enhance the properties of the deodorant.

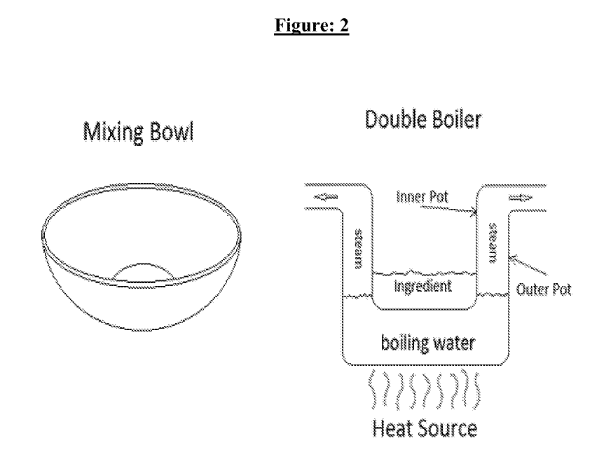

FIG. 2 shows the double boiler that is required for cooking or manufacturing the desired deodorant product. The double boiler as shown is a set of two fitted saucepans or pots that are stacked together with space between them. The bottom saucepan is filled with water, and then the second saucepan is stacked on top. The heating source causes the steam and boiling water from the bottom pot to raise the temperature of the upper pot to 100 degrees Celsius.

The steps included in the process of manufacturing the deodorant formulation in accordance with the present invention are outlined as follows:

FIG. 3 is an overview of the first part of the process of the deodorant formulation using the particular ingredients listed in FIG. 1. The subject deodorant compound can be manufactured by heating 6 ounces of coconut oil in the double boiler containing water that has been brought to a boil at normal atmospheric pressure. After the coconut oil reaches the temperature of 100 degrees Celsius, 1.5 ounces of candelilla wax and 0.5 ounce of any desired fragrance are added therein termed as “Mix: 1”). The candelilla wax and optional fragrance are allowed to completely dissolve within the coconut oil, maintained at a temperature of 100 degrees Celsius inside the double-boiler. This results in an oily mixture of coconut oil, candelilla, and optional fragrance (termed as “mix: 2”)

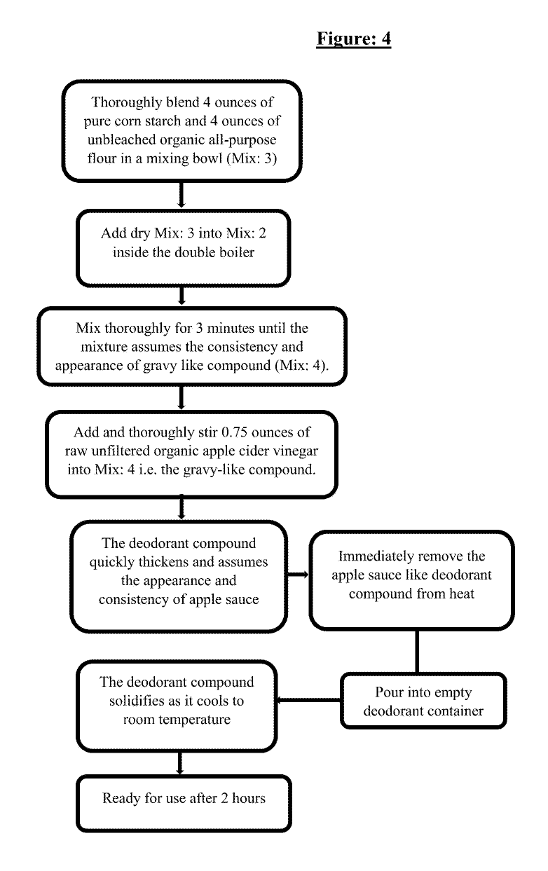

FIG. 4, which shows the second part comprising the process of manufacturing the deodorant compound, runs in continuation to first part as previously stated. In a mixing bowl, 4 ounces of pure corn starch is thoroughly mixed with 4 ounces of unbleached organic all-purpose flour to form a dry mixture (termed as “Mix 3”). After the candelilla wax has been completely dissolved within the coconut oil, the dry mixture of pure corn starch and unbleached organic all-purpose flour (Mix: 3) is then added to the Mix 2 i.e. oily mixture of coconut oil, candelilla, and optional fragrance inside the double boiler. As the ingredients inside the double boiler are mixed thoroughly and continuously for a period of three minutes, they assume the consistency and appearance of a gravy-like compound (termed as “Mix 4”). Following this, 0.75 ounces of raw unfiltered organic apple cider vinegar is finally added to and thoroughly mixed into the gravy-like compound of Mix: 4. The deodorant compound quickly thickens and assumes the appearance and consistency of apple sauce. The apple sauce-like deodorant compound is removed from the boiler and is poured immediately into deodorant containers. After cooling to room temperature for two hours, the deodorant is ready for use.

The desired deodorant composition can only be attained by strictly adhering to the timing, quantity and mixing sequence of the compounds per the above disclosed embodiment. In addition to providing a mild, pleasant natural fragrance to the deodorant compound, the usage of coconut oil prevents the obtained final product from drying out and thus retains a form that is suitable for application to the underarms. Inventions in prior art use sheer butter for the said purpose, but it has been found that its usage tends to dry out over time and cause skin irritation, and is, therefore, not a suitable ingredient in the deodorant composition. Further, because it was discovered that elevated blood pressure readings resulted after several days of using prior versions of the composition that contained sodium bicarbonate, sodium bicarbonate was substituted with unbleached organic all-purpose flour. The integration of candelilla wax into the invention provided the firmness that was lacking in the deodorant compound. Thus, the final product effectively eliminates odors and maintains both its moisture and structural integrity without causing skin irritation.

The invention's simple composition and the equally simple process through which it is created set it apart from other compositions known in the prior art. The invention is the most effective all-natural deodorant available with so few ingredients. Additionally, the deodorant can be stored between 0 and 32 degrees Celsius indefinitely without becoming rancid or losing its efficacy.

Similar embodiments of the present invention can be created by substituting ingredients of similar properties for those used in the present invention. For example, those either allergic to or that have an aversion to coconut oil can use, without limitation, another natural oil such as olive oil, almond oil, or grapeseed oil. Similarly, based on the user's preference, choice of fragrance can be made, without limitation, among lavender, musk, gardenia, clary sage and vanilla.

In yet another embodiment of the present invention, the deodorant is prepared without adding fragrance and by substituting grapeseed oil for coconut oil since grapeseed oil has similar properties to coconut oil, yet has virtually no fragrance.

This invention stands out from all the other available natural deodorants because it is unique in that it contains so few ingredients, because it uses the simplest method of preparation of a topical deodorant, and because it retains efficacy both in terms of structural integrity and in eliminating underarm odor.

Due to the prominence of coconut oil, the present invention not only has antibacterial properties that kill odor-producing bacterial, but it also produces soothing, moisturizing effects on skin, which prevents skin irritation. Additionally, the ingredients of the present invention are recognized as safe for human contact, as they are all widely used in the food industry.

1. A topical deodorant composition made with 100% vegan ingredients. The deodorant composition comprises of,
a) Pure corn starch;
b) unbleached organic all-purpose flour;
c) Virgin Coconut oil;
d) Candelilla Wax;
e) Raw unfiltered organic apple cider vinegar;
f) Fragrance.

2. The topical deodorant composition of claim 1, wherein the ingredients comprises of the following parts by volume of the overall deodorant composition:
a) 8 parts of Pure Corn starch;
b) 8 parts of unbleached organic all-purpose flour;
c) 12 parts of Virgin Coconut oil;
d) 6 parts of Candelilla wax;
e) 3 parts of raw unfiltered organic apple cider vinegar;
f) 2 parts of fragrance.

3. The topical deodorant composition of claim 1, where the composition comprises of the following approximate percentages:
a) of about 20.5% of pure Corn Starch;
b) of about 20.5% of Unbleached organic all-purpose flour;
c) of about 30.8% of Virgin Coconut Oil;
d) of about 15.4% of Candelilla Wax;
e) of about 7.7% of raw unfiltered organic apple cider vinegar;
f) of about 5.1% of Fragrance;

4. The topical deodorant composition of claim 1, wherein the virgin coconut oil is substituted with other available natural oil having similar characteristic, such natural oil without limitation includes olive oil, almond oil, or grape seed oil.

5. The topical deodorant composition of claim 1, wherein the choice of fragrance without limitation includes lavender, musk, gardenia, clary sage and vanilla.

6. The topical deodorant composition of claim 1, wherein the said deodorant composition is manufactured fragrance free by only substituting Virgin Coconut oil with Grape seed oil.

7. A method of preparing a topical deodorant composition made with 100% vegan ingredients. The method comprising of:
a) Heating virgin coconut oil in a double boiler containing water, wherein the water in the double boiler is brought to boil at normal atmospheric pressure;
b) Adding Candelilla wax and any desired fragrance to the coconut oil when it reaches the temperature of 100 degrees Celsius;
c) Stirring the mixture of coconut oil, Candelilla wax and fragrance until the Candelilla wax is completely dissolved within the coconut oil;
d) Thoroughly mixing in a separate bowl pure Corn starch with unbleached organic all-purpose flour;
e) Adding the dry mixture of pure corn starch and unbleached organic all-purpose flour to the oily mixture of Coconut oil, Candelilla wax and fragrance;
f) Stirring the ingredients inside the double boiler vigorously for at least three minutes until it assumes the consistency and appearance of a gravy-like compound;
g) Adding and thoroughly stirring raw unfiltered organic apple cider vinegar into the gravy-like compound of all other blended ingredients; wherein the deodorant compound thickens and assumes the appearance and consistency of apple sauce;
h) The deodorant compound is removed from the boiler and is poured immediately into deodorant containers.
i) The deodorant compound solidifies after two hours of cooling down to room temperature.

8. The method of claim 7, wherein the said ingredients comprises of the following precise portions by volume:
a) of about 6 ounces of Virgin Coconut Oil;
b) of about 1.5 ounces of Candelilla wax;
c) of about 0.5 ounces of Artificial fragrance;
d) of about 4 ounces of Pure Corn Starch;
e) of about 4 ounces of unbleached organic all-purpose flour;
f) of about 0.75 ounce of raw unfiltered organic apple cider vinegar.

9. The method of claim 7, wherein the said deodorant compound is poured into a deodorant container selected from a group consisting of a squeezable tube, dispenser, roll-on bottles, rigid tube or pot.
